(19) United States
(12) Patent Application Publication
(10) Pub. No.: US 2024/0172951 A1
(43) Pub. Date: May 30, 2024

(54) Method and Device for detecting Information of Object In Room, Storage Medium and Processor

(71) Applicant: BEIJING TSINGRAY TECHNOLOGY CO., LTD., Beijing (CN)

(72) Inventors: Yuguo DING, Beijing (CN); Hao WU, Beijing (CN); Wei RAO, Beijing (CN); Pingzhen SHANG, Beijing (CN); Hongjun BAN, Beijing (CN); Siliang CHEN, Beijing (CN); Shuai SHAO, Beijing (CN)

(21) Appl. No.: 17/783,002

(22) PCT Filed: Jul. 14, 2021
(86) PCT No.: PCT/CN2021/106310

§ 371 Date: Jan. 19, 2023

(51) Int. Cl. A61B 5/05; A61B 5/11; G01S 7/00; G01S 7/41

(52) U.S. Cl. CPC A61B 5/05; A61B 5/1117; G01S 7/003; G01S 7/415; A61B 2505/07

(57) ABSTRACT

The application discloses a method and device for detecting information of an object in a room, a storage medium and a processor. The method comprises the following steps: collecting target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears; analyzing behavior state information of the target object based on the target information; and displaying the behavior state information of the target object on a display interface. Through the application, the problem of poor information detection effect on indoor personnel in related technologies is solved.

### TECHNICAL FIELD

The application relates to the technical field of information processing, in particular to a method and device for detecting information of an object in a room, a storage medium and a processor.

### BACKGROUND

At present, in related technologies, watches or bracelets judge the vital signs of users in home scenes by measuring blood oxygen and heart rate. However, users need to wear it all the time to generate vital sign data. Due to the influence of people sweating and bathing, this kind of equipment may be abandoned after being taken off by users. And users may feel uncomfortable after wearing the device; Or when the strap or bracelet is loose, the contact between the device and the skin is not close, which will lead to a large error between the test data and the real value. All of the above are the defects of contact measuring equipment.

At present, there is a polysomnography monitor in the related technology, which can acquire the electrocardiogram and physiological data of people by means of electrodes, and then extract vital signs such as heart rate and respiratory rate. The defect of this kind of equipment is that it is too big and heavy to be portable. And although this kind of equipment is the gold standard for vital signs monitoring, it is expensive and cannot be applied to the general public.

At present, in related technologies, there is also a smart camera, which can monitor the user's behavior rules by means of image recognition. The defect of this kind of equipment is that it infringes users' privacy, and users reject the use of this kind of equipment in home scenes, especially in bedrooms and bathrooms. Image processing requires a processor with higher frequency, higher power consumption and higher price.

At present, in related technologies, there are infrared monitoring devices, which can sense the temperature of human body by infrared pyroelectricity, so as to obtain whether there is life in a certain range. Although the cost of this kind of equipment is very low, its disadvantage is that it is insensitive to distance and can not accurately know the location and signs of users. Moreover, the angle of field of view monitored by Fresnel lens is generally ±40°, and at least half of the angle space is the dead angle of infrared monitoring. Infrared equipment is also greatly affected by temperature and environment, and monitoring failure will occur in high temperature environment and dense fog environment.

At present, in related technologies, there is also a big data intelligent analysis system using water meter or electricity meter, which has the advantage of roughly analyzing and evaluating the simplest state of users with the help of existing data. However, the defect of this kind of system is that the data is too thin to know the daily life behavior and continuous and detailed vital signs of users deeply.

At present, there is no effective solution to the problem of poor information detection for indoor personnel in related technologies.

### SUMMARY

The main purpose of this application is to provide a method and device for detecting information of an object in a room, a storage medium and a processor, so as to solve the problem of poor information detection effect for indoor personnel in related technologies.

In order to achieve the above object, according to one aspect of the application, the method for detecting information of an object in a room is provided. The method comprises the following steps: collecting target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth; analyzing behavior state information of the target object based on the target information; and displaying the behavior state information of the target object on a display interface.

Further, before collecting the target information of the target object in the room by the target detection equipment, the method further comprises: arranging a plurality of radar monitoring devices in the room, wherein the plurality of radar monitoring devices are configured to collect falling behavior information and indoor behavior information of the target object; and taking the plurality of radar monitoring devices as devices in the target detection equipment.

Further, after the target information of a target object in the room is collected by target detection equipment, the method further comprises: uploading the target information to the cloud server by the WIFI module; analyzing the behavior state information of the target object based on the target information includes analyzing the target object based on the target information in the cloud server to obtain the behavior state information of the target object.

Further, the target information also includes the location information of the target object. In the cloud server, the target object is analyzed based on the target information to obtain the behavior state information of the target object, which includes: when the location information of the target object indicates that the target object is in an indoor bedroom bed, analyzing the vital sign information and behavior state information of the target object by using the behavior information in the cloud server; when the position information of the target object indicates that the target object is in the indoor bathroom, analyzing the behavior state information of the target object by using the behavior information in the cloud server; when the location information of the target object indicates that the target object is in the indoor living room, the vital sign information is used in the cloud server to analyze the behavior state information of the target object.

Further, after analyzing the behavior state information of the target object by using the behavior information in the cloud server, the method further includes: when the behavior state information indicates that the target object has a falling behavior phenomenon, triggering the first reminding information; when the behavior state information indicates that the vital signs of the target object are abnormal, triggering second reminding information; when the behavior state information indicates that the bed leaving time of the target object exceeds a preset time, triggering a third reminding message.

Further, the method further comprises: acquiring a set of target information of the target object in a preset time period from a cloud server; screening data information of multiple dimensions from the set of target information; evaluating the first health data of the target object based on the preset weight of each dimension and the data information of each dimension, wherein the health data at least comprises the body state information of the target object and the prediction information of the body potential risk.

Further, after evaluating the health status and body risk of the target object based on the preset weight of each dimension and the data information of each dimension, the method further comprises: displaying the first health data on the display interface according to a preset display form.

Further, after collecting the target information of the target object in the room by the target detection device, the method further comprises: analyzing whether the target object is in a moving state based on the behavior information in the target information; when the target object is in a moving state, the movement vitality information of the target object is determined.

Further, the target information also includes the position information of the target object. After collecting the target information of the target object in the room by the target detection device, the method further includes: analyzing whether the target object is in bed based on the position information in the target information; when it is analyzed that the target object is in bed, judging whether the target object is in a sleep state based on vital sign information in the target information; analyzing the heartbeat data and breathing data of the target object when it is judged that the target object is in a sleep state; generating a sleep analysis report of the target object based on the heartbeat data and respiratory data of the target object, wherein the sleep analysis includes at least the sleep quality information of the target object and the prediction information of the chronic respiratory disease of the target object.

In order to achieve the above object, according to another aspect of the application, there is provided the device for detecting information of an object in a room. The device comprises a first collecting unit, configured to collect target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth; a first analysis unit, configured to analyze the behavior state information of the target object based on the target information; and a first display unit, configured to display the behavior state information of the target object on a display interface.

Further, the device further comprises a first arranging unit, configured to arrange a plurality of radar monitoring devices in the room, before collecting the target information of the target object in the room by the target detection equipment, wherein, the plurality of radar monitoring devices are configured to collecting falling behavior information and indoor behavior information of the target object; a first determination unit configured to take the plurality of radar devices as devices in the target detection equipment.

Further, the device further comprises a first arranging unit, configured to arrange a plurality of radar monitoring devices in the room, before collecting the target information of the target object in the room by the target detection equipment, wherein the plurality of radar monitoring devices are configured to collect the falling behavior information and indoor behavior information of the target object a third analysis unit configured to analyze the target object based on the target information in the cloud server to obtain the behavior state information of the target object.

Further, the target information also includes the location information of the target object, and the third analysis unit includes: a first analysis module configured to analyze the vital sign information and behavior state information of the target object in the cloud server by using the behavior information when the location information of the target object indicates that the target object is in the indoor bedroom bed; a second analysis module, configured to analyze the behavior state information of the target object in the cloud server by using the behavior information when the location information of the target object indicates that the target object is in the indoor bathroom; a third analysis module, configured to analyze the behavior state information of the target object in the cloud server by using the vital signs information when the location information of the target object indicates that the target object is in the living room indoors.

Further, the device further comprises a first triggering unit, configured to trigger first reminding information when the behavior state information indicates that the target object has a falling behavior phenomenon after analyzing the behavior state information of the target object by using the behavior information in the cloud server; a second triggering unit configured to trigger second reminding information when the behavior state information indicates that the vital signs of the target object are abnormal; a third triggering unit, configured to trigger a third reminding message when the behavior state information indicates that the bed leaving time of the target object exceeds a preset time.

Further, the device further comprises a first acquisition unit configured to acquire the set of target information of the target object in a preset time period from the cloud server; a first filtering unit configured to filter out data information of multiple dimensions from the set of target information; a first evaluation unit, configured to evaluate the first health data of the target object based on the preset weights of each dimension and the data information of each dimension, wherein the health data at least comprises the body state information of the target object and the prediction information of the potential risks of the body.

Further, the device further comprises a second display unit, which is configured to display the first health data on the display interface according to a preset display form after evaluating the health status and body risk of the target object based on the preset weights of each dimension and data information of each dimension.

Further, the device further includes a fourth analysis unit configured to analyze whether the target object is in a moving state based on the behavior information in the target information after the target information of the target object in the room is collected by the target detection device; a first determining unit configured to determine the movement vitality information of the target object when the target object is in a moving state.

Further, the target information also includes the location information of the target object, and the device further includes a fifth analysis unit configured to analyze whether the target object is in bed based on the location information in the target information after collecting the target information of the target object in the room by the target detection device; a first judging unit configured to judge whether the target object is in a sleep state based on vital signs information in the target information when it is analyzed that the target object is in bed; a sixth analysis unit configured to analyze the heartbeat data and respiratory data of the target object when it is judged that the target object is in a sleep state; a first generating unit configured to generate a sleep analysis report of the target object based on the heartbeat data and respiratory data of the target object, wherein the sleep analysis includes at least the sleep quality information of the target object and the prediction information of the chronic respiratory disease of the target object.

In order to achieve the above object, according to another aspect of the application, there is provided a processor for running a program, wherein the program executes the method for detecting information of an object in a room as described in any one of the above.

In order to achieve the above object, according to another aspect of the application, there is provided a storage medium including a stored program, wherein the program executes the method for detecting information of an object in a room as described in any one of the above.

The application adopts the following steps: collecting target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth; analyzing behavior state information of the target object based on the target information; and displaying the behavior state information of the target object on a display interface, solving the problem of poor information detection effect of indoor personnel in related technologies. By analyzing the target information including vital signs information and behavior information of the target object, the behavior state information of the target object can be accurately determined and displayed on the display interface, which is convenient for users to visually view the behavior state information of the target object, thus improving the user experience and further achieving the effect of improving the information detection effect of indoor personnel.

### BRIEF DESCRIPTION OF THE DRAWINGS

The drawings, which form a part of this application, are used to provide a further understanding of this application. The illustrative embodiments and descriptions of this application are used to explain this application, and do not constitute undue restrictions on this application. In the drawings:

FIG. 1 is a flow chart of the method for detecting information of an object in a room provided according to an embodiment of the present application;

FIG. 2 is a schematic diagram of the method for detecting information of an object in a room provided according to an embodiment of this application;

FIG. 3 is a diagram of equipment installation and deployment of the present invention;

FIG. 4 is a schematic diagram of the installation of the living room detection radar in the embodiment of this application;

FIG. 5 is a schematic diagram of the detection angle of the living room detection radar in the embodiment of this application;

FIG. 6 is a schematic diagram of the detection area of the vital sign monitoring radar in the embodiment of this application;

FIG. 7 is a schematic diagram of the data format of the living room detection radar in the embodiment of this application;

FIG. 8 is the connection diagram of components and equipment in the embodiment of this application;

FIG. 9 is a system diagram of the docking cloud service in the embodiment of this application;

FIG. 10 is a flowchart of data integration and analysis processing in the embodiment of this application;

FIG. 11 is a network front-end service architecture diagram in the embodiment of this application;

FIG. 12 is a vital sign data presentation diagram in the embodiment of the present application;

FIG. 13 is a schematic diagram of abnormal triggering alarm in the embodiment of this application;

FIG. 14 is a data presentation diagram of behavior law in the embodiment of the present application;

FIG. 15 is a situation presentation diagram in the embodiment of this application;

FIG. 16 is a schematic diagram of data mining value in the embodiment of this application;

FIG. 17 is a schematic diagram of the device for detecting information of an object in a room provided according to an embodiment of the present application.

### DETAILED DESCRIPTION OF THE EMBODIMENTS

It should be noted that the embodiments in this application and the features in the embodiments can be combined with each other without conflict. The application will be described in detail with reference to the drawings and examples.

In order to make people in the technical field better understand the application scheme, the technical scheme in the application embodiment will be clearly and completely described below with reference to the drawings in the application embodiment. Obviously, the described embodiment is only a part of the application embodiment, not all the embodiments. Based on the examples in this application, all other examples obtained by ordinary technicians in the field without creative work should belong to the scope of protection in this application.

It should be noted that the terms “first”, “second” and so on in the specification and claims of this application and the above drawings are used to distinguish similar objects, but not necessarily to describe a specific order or sequence. It should be understood that the data thus used can be interchanged under appropriate circumstances to facilitate the embodiments of the present application described herein. In addition, the terms “including” and “having” and any variations thereof are intended to cover non-exclusive inclusion, for example, a process, method, system, product or equipment containing a series of steps or units need not be limited to those steps or units explicitly listed, but may include other steps or units not explicitly listed or inherent to these processes, methods, products or equipment.

According to the embodiment of the application, the method for detecting information of an object in a room is provided.

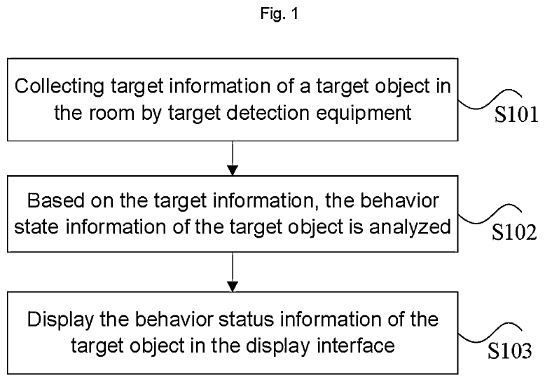

FIG. 1 is a flow chart of the method for detecting information of an object in a room according to an embodiment of the present application. As shown in FIG. 1, the method includes the following steps: S101, collecting target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth.

For example, the target detection equipment can be vital signs monitoring radar, fall detection radar in bathroom and detection radar in living room, which are used to collect indoor behavior information of target object in the room, and the behavior information can include whether the target objects are in bed or in motion. The vital sign monitoring equipment worn by the target, for example, the dynamic blood oxygen detector hung on the ear or finger of the tested person, the bracelet equipment worn on the wrist, etc., is used to collect the indoor vital sign information of the indoor target, which may include the respiratory rate and heart rate of the target.

S102, analyzing the behavior state information of the target object based on the target information.

For example, by the above target detection devices, target object in the room are collected with target information, and data are generated and output, including data output of living room detection radar, data output of fall detection radar in bathroom and data output of vital sign detection radar. Then, the output data uploads the target information to the cloud server by the WIFI module and/or Bluetooth module. After that, all data are mixed together in the cloud database and then mixed together and processed by the cloud computing server to analyze the behavior status information of the target object.

S103, displaying the behavior state information of the target object on the display interface.

For example, after the data of the living room radar is mixed together and processed, the real-time coordinates of the person under test in the room will be displayed in the interface, so that the Client Personnel can intuitively see the state and behavior of people in the home environment (corresponding to the above-mentioned behavior state information). After the data of vital signs monitoring radar is mixed together and processed, the respiratory rate, respiratory waveform, heart rate, heartbeat waveform and sleep state of the person in the room can be output in real time. After the data of the fall detection radar in bathroom is mixed together and processed, it will show the abnormal alarm state of the actual people falling in the bathroom by a cartoon character falling. At the same time, the client will highlight the abnormal emergency state by means of sound, so that the Client Personnel can recognize it audibly. The processing after data integration pays more attention to the analysis and evaluation of people's health and vitality in the house. The contents of the exhibition include: statistics of sitting time, standing time, sleeping time, walking time, toilet time and frequency, etc. Furthermore, the invention provides a data disk for all displayed information, and by the data disk, all family states where the integrated home health monitoring system is installed can be visually observed. The data can be displayed in the WEB client and the mobile APP client. Multi-dimensional data information is screened out from the data information after data integration and analyzed, so as to evaluate the first health data of the target object, which should at least include the health status data and potential risk prediction data of the target object.

By the above steps S101 to S103, analyzing the target information including vital signs information and behavior information of the target object can accurately determine the behavior state information of the target object, and display it on the display interface, which is convenient for users to visually check the behavior state information of the target object, thereby improving the user experience and further achieving the effect of improving the information detection effect of indoor personnel.

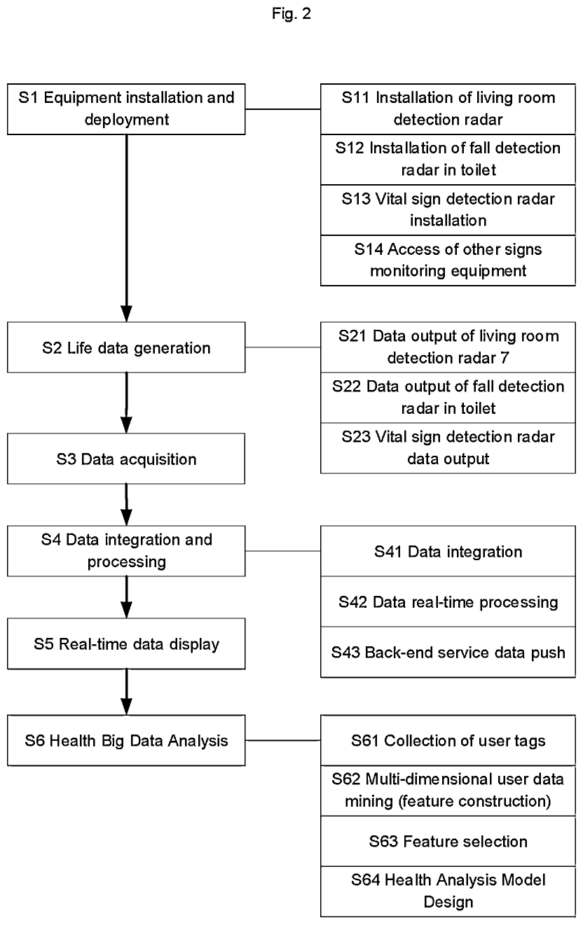

As shown in FIG. 2, firstly, multiple detection devices do edge calculation in their own processors to output real-time signs and behavior data to the cloud; cloud data storage module, relational database and cloud computing kernel participate in multi-dimensional data integration, analysis, judgment, upgrade and other processing processes, and push the pre-presented data to the client for display; in addition to providing visual presentation of the situation, the client also has the function of information feedback, which is convenient for correcting and iteratively upgrading the analysis algorithm. The specific method is shown in steps S1-S6.

Before collecting the target information of the target object in the room by the target detection device, the method further comprises: arranging a plurality of radar monitoring devices in the room, wherein the plurality of radar monitoring devices are configured to collect falling behavior information and indoor behavior information of the target object; and taking the plurality of radar monitoring devices as devices in the target detection equipment. By the installed and deployed radar monitoring equipment, the target information of target object in the room can be accurately collected. Radar monitoring equipment can include vital signs monitoring radar, fall detection radar in toilet and living room detection radar.

Step S1, Equipment installation and deployment.

Install the vital sign monitoring radar, fall detection radar in bathroom and living room detection radar on the wall and supply power. After other vital signs monitoring devices are connected to the device which can be used for data transfer (the device is usually vital signs monitoring radar), other vital signs monitoring devices are put on the body of the tested person.

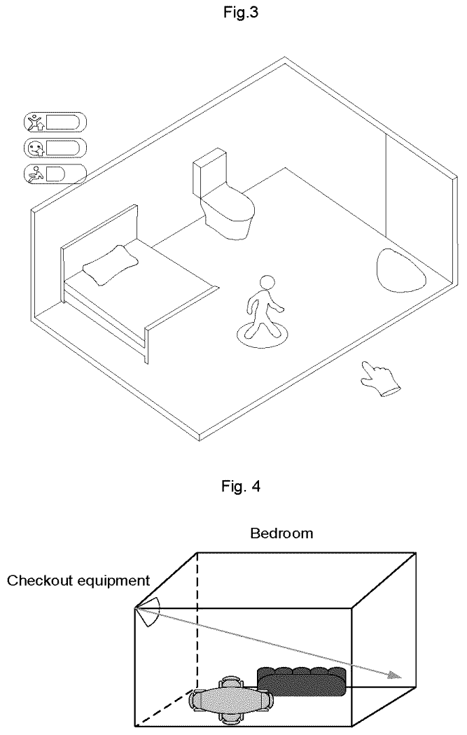

The installation location of radar monitoring equipment is shown in FIG. 3, in which the square equipment is the installation equipment. Equipment installation and deployment are detailed in steps S11-S14.

Step S11, Detection radar installation in living room.

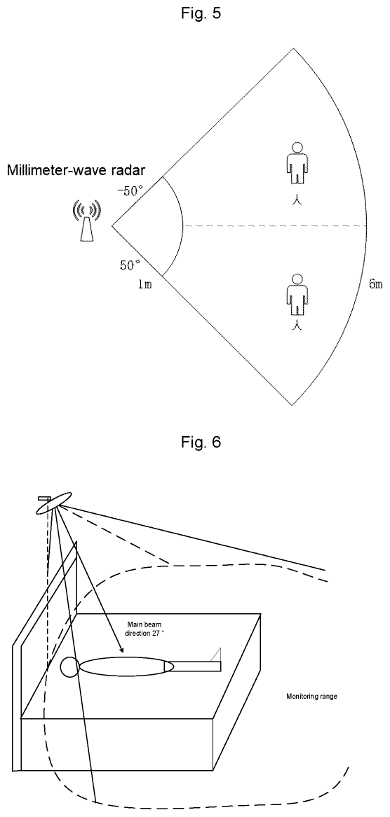

The living room radar can be installed in a corner of the ceiling of the living room, and the normal line of the front surface of the equipment is aligned with the corner of the living room (diagonal line of the installation position). As shown in FIG. 4, the antenna beam angle of millimeter wave radar equipment is 50°, which is larger than the corner angle of normal room by 90°. As shown in FIG. 5, the distance coverage is more than 8 m, which can cover most living rooms.

Step S12, installing the fall detection radar in toilet.

The fall detection radar in toilet can be installed on the ceiling directly above the toilet. The normal of the front surface of the equipment is perpendicular to the ground, and the radar equipment provides a wide-angle antenna of 60°, and the monitoring range of the equipment can cover most toilets.

Step S13, Installation of vital signs monitoring radar.

The vital sign monitoring radar can be installed on the wall of the bedside. As shown in FIG. 6, the detection radar equipment is installed on the wall between 1 m and 1.3 m from the bed height; The equipment can also be installed on the ceiling directly above the bed surface. The detection range of the equipment can just cover the whole bed surface.

The radar monitoring equipment disclosed in the above steps S11-S14 has a wide coverage, and can monitor the vital signs and behavior rules of residents in the whole house without dead ends. The living room detection radar can track any position of the user in the living room. Once the user enters the bedroom and bathroom, the right to know the user's status data is transferred to the vital signs monitoring radar or bathroom detection radar. Thereby realizing real-time and accurate acquisition of target information of users in any corner of the whole house environment.

Step S14, access of other sign detection equipment.

The above-mentioned radar monitoring equipment can be connected with other vital sign detection equipment by Bluetooth transmission, such as wearable blood oxygen detection equipment and heart rate detection equipment. These equipment will regularly transmit blood oxygen data and heart rate data to the vital sign monitoring radar by Bluetooth communication, and complement each other with radar vital sign data to ensure that the vital information of the tested person is comprehensive and reliable.

Step S2, life data generation.

The data generated by all the devices in this application have a fixed protocol and format, and the back-end server analyzes and processes the data according to the protocol content of each type of data. The specific steps of life data generation are shown in steps S21-S23.

Step S21, the living room detects the radar data output.

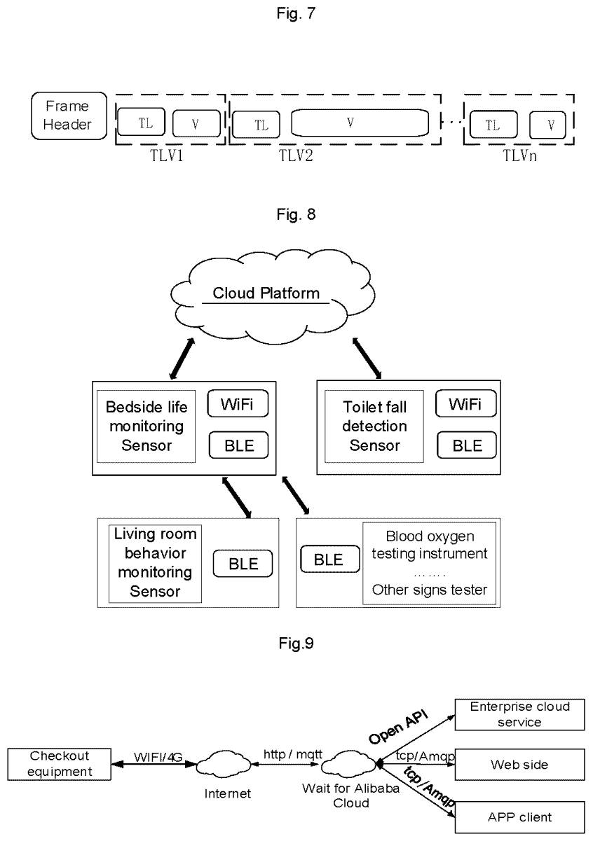

The data format of the living room detection radar is shown in FIG. 7. The first part is the data header, and the rest is the point cloud information and target information. The specific content is defined as follows:

TLV1 is the output of point cloud data, which includes the location and number of point clouds, and each point cloud contains the information of point traces.

TLV2 is the target data output, which includes the coordinate information of indoor moving objects, the speed information of people, the acceleration information of people and the attitude information of people.

TLV3 is the target classification output, and the output content includes the type of each target, by which adults, children, animals, moving robots or other objects can be distinguished.

Among them, the information of point cloud reflects the information of moving objects in space indiscriminately, and can preliminarily distinguish the volume and position of the target. The target information is the result of equipment classification processing, which can distinguish the types of targets, coordinates, trajectory, speed, acceleration, attitude and so on.

Step S22, data output of fall detection radar in toilet.

The data format of fall detection radar in toilet also consists of its own data header and data content. There is a code word identifying the type of this device in the data header, and the specific data content is defined as shown in Table 1 below.

TABLE 1

| Output Data Content | Type and Length | Remarks |
|---|---|---|
| upload.range_profile_macro_pow_dB | uint16 * 64 | One-dimensional range profile |
| | | on Macro scale |
| upload.range_profile_micro_pow_dB | uint16 * 64 | One-dimensional range profile |
| | | on Micro scale |
| upload.flag_target_exist | uint8* 1 | Target presence flag (0 = does |
| | | not exist, 1 = exists) |
| upload.radar_install_height | uint8* 1 | Radar installation height |
| upload.flag_fall_down_L1 | uint8* 1 | L1 falling sign (0 = not falling, |
| | | 1 = falling) |
| upload.flag_fall_down_L2 | uint8* 1 | L2 falling sign (0 = not falling, |
| | | 1 = falling) |
| upload.fall_score | uint16* 1 | Fall score (full score follows |
| | | fall response time) |
| upload.body_envelope_fall_score | uint16* 1 | Body spectrum width fall score |
| upload.body_energy_fall_score | uint16* 1 | Physical energy fall score |
| upload.false_alarm_rate | uint8 * 1 | Early warning sensitivity of |
| | | falling score judgment |
| upload.len_fall_statistics | uint8* 1 | Fall detection duration, unit: |
| | | seconds |

Among them, one-dimensional range profile on Macro scale can identify the big movements of indoor people, and one-dimensional range profile on Micro scale can identify the weak movements of indoor people. The fall sign is used as the basis for judging whether the indoor person falls.

Step S23, vital signs monitoring radar data output.

The data format of vital sign monitoring radar also consists of its own data header and data content. There is a code word identifying the type of this device in the data header, and the specific data content is defined as shown in Table 2 below.

TABLE 2

| | Byte | | |
|---|---|---|---|
| Name | Bit | Data Type | Detailed Annotation |
| Frame number bit | 0-3 | uint32_t | Accumulating from |
| | | | 0 indicates |
| | | | data continuity. |
| Respiratory phase | 4-23 | uint16_t * 10 | |
| data | | | |
| Heartbeat phase | 24-43 | uint16_t * 10 | |
| data | | | |
| Breathing BPM | 44 | uint8_t | |
| Heartbeat BPM | 45 | uint8_t | |
| S1 state | 46 | uint8_t | 0~9 countdown |
| | | | 10-0xa nobody |
| | | | 11-0xb too close |
| | | | 12-0xc mismatch |
| | | | 13-0xd coordination |
| S2 state | 47 | uint8_t | 0-out of bed |
| | | | 1-Awake |
| | | | 2-Shallow sleep |
| | | | 3-Deep sleep |
| | | | 4-Turn over |
| One-dimensional | 88-123 | uint8_t * 36 | |
| range profile | | | |
| Depth of respiration | 124 | uint8_t | |
| Distance dimension | 125 | uint8_t | |
| power strong point | | | |
| index | | | |
| Distance dimension | 126 | uint8_t | |
| power peak | | | |
| Noise power | 127 | uint8_t | |
| Target distance | 129 | uint8_t | |
| gate | | | |
| Out of bed and | 130-131 | uint16_t | |
| other abnormal | | | |
| conditions | | | |
| Out of bed time (s) | 132-133 | uint16_t | |

Among them, breathing and heartbeat phase data can be used to analyze the breathing patterns and heart vitality of people in bed. And breathing and heartbeat BPM can most intuitively display the number of breaths and heartbeats per minute of people in bed. The S1 state in table 2 shows the active state of people in bed, and the S2 state shows whether people are in bed and the sleep state when they are in bed. Other information, as the auxiliary data of vital signs analysis, can comprehensively analyze people's sleep quality and chronic disease symptoms.

Step S3, cloud data.

As shown in FIG. 8, vital signs monitoring radar and fall detection radar in toilet have a large amount of data, and these two devices are equipped with wifi module and Bluetooth module at the same time. The data of the testee's sleep process is directly uploaded to the cloud by the wifi module of vital signs monitoring radar, and the data of the testee's falling state and behavior law in the bathroom are also uploaded to the cloud by its own wifi module. The data volume and refresh rate of the living room radar and other vital signs detection devices are relatively small. The data is first transmitted to the processor of vital signs monitoring radar by Bluetooth communication, and then packaged and uploaded to the cloud by wifi module. That is, after the target information of the target object in the room is collected by the target detection device, the method further includes: uploading the target information to the cloud server by the WIFI module; analyzing the behavior state information of the target object based on the target information includes: analyzing the target object based on the target information in the cloud server to obtain the behavior state information of the target object. In the cloud, the target data can be processed and analyzed in real time, so as to achieve real-time monitoring of the target object, and when there is an abnormal situation, an alarm will be given immediately.

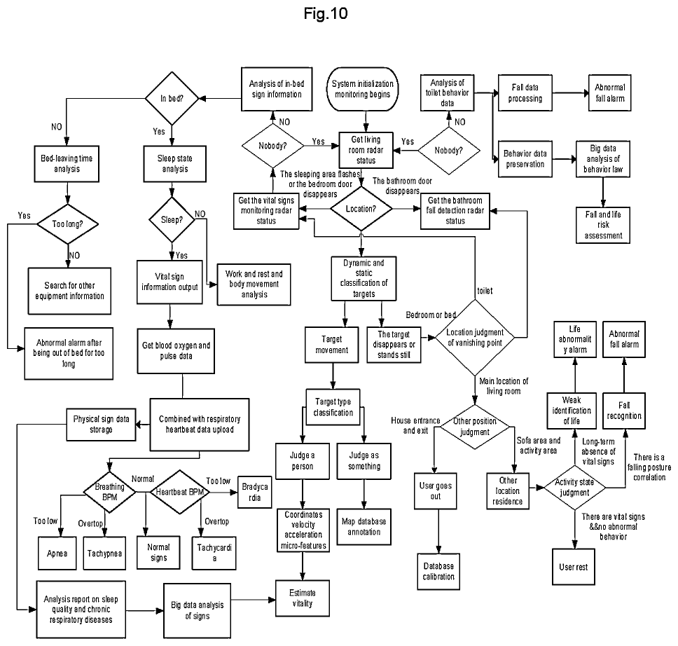

As shown in FIG. 9, the detection device can complete the access of data to the Internet by sending AT instructions to the wifi module by the main control chip, and the data access function can also be realized by the 4G module. Every second, the device uploads a packet of data, and the data is connected with Internet of Things platforms such as Alibaba Cloud by http protocol or MQTT protocol. Finally, the data will be presented in the enterprise private cloud, public WEB or APP client, and the system will push the data from the cloud platform to the client by the Open API interface or AMQT protocol.

Step S4, data integration and processing.

All the data in this application are mixed together in the database in the cloud and then processed by the cloud computing server. The specific steps of data integration and processing can be shown in steps S41-S43.

Step S41, data integration.

In this application, data of multiple dimensions will be generated, and in order to ensure the most effective utilization of data, a set of perfect data integration process is needed to comprehensively judge and locate key data nodes. The back-end server efficiently analyzes the data of key nodes, so as to locate the state information of the testee.

Optionally, the target information in the embodiment of this application also includes the location information of the target object. In the cloud server, the target object is analyzed based on the target information to obtain the behavior state information of the target object, which includes: when the location information of the target object indicates that the target object is in an indoor bedroom bed, analyzing the vital sign information and behavior state information of the target object by using the behavior information in the cloud server; when the position information of the target object indicates that the target object is in the indoor bathroom, the behavior information is used in the cloud server to analyze the behavior state information of the target object; when the location information of the target object indicates that the target object is in the indoor living room, the vital signs information is used in the cloud server to analyze the behavior state information of the target object.

By analyzing the position information of the target object by the above steps, the behavior state information of the target object can be accurately determined, and the accuracy of the data information can be ensured, thus improving the user experience and further achieving the effect of improving the information detection of indoor personnel.

As shown in FIG. 10, after the system is initialized, the back-end server first acquires the data of the living room radar, and determines the type of acquired equipment data according to the analysis of the target position. When the position of the target object disappears at the door of the bedroom, it is transferred to obtaining the data of vital signs radar (corresponding to the above vital signs information) for analysis; when the position of the target disappears at the entrance of the bathroom, it is transferred to obtaining the data of the fall detection radar in bathroom (corresponding to the above behavior information) for analysis; Otherwise, give priority to analyzing and processing radar data of living room. According to the radar data, it is further judged whether there is no one in the target location area; when there is no one, the radar status of the living room will be acquired again; when there is one, the information of the person's physical signs in bed will be judged; when it is judged that the person has not left the bed, the sleep state of the person will be analyzed; when the vital signs monitoring radar confirms that the person is asleep, the blood oxygen and pulse data of other wearable devices will be transferred by the Bluetooth module, and the respiratory heartbeat data generated by the vital signs monitoring radar will be mixed together and uploaded to the back-end server. And judge whether the breathing BPM index of this person is normal, when the breathing BPM is normal, prove that the vital signs of this person are normal, and further judge whether the heartbeat BPM is normal; when the heartbeat BMP is too low, it proves that this person's heart skips slowly; when the heartbeat BMP is too high, it proves that this person's heart skips slowly; when the respiratory BPM is too high, it means that the person is short of breath; when the respiratory BPM is too low, it means that the person is apnea; after judging the person's vital signs, store the relevant data of vital signs, generate the analysis report of the person's sleep quality and chronic respiratory diseases, analyze the big data of vital signs, estimate the person's vitality, analyze the person's sleep state, and analyze the person's work schedule and body movement when he doesn't fall asleep; when it is judged that the person has left the bed, the time of leaving the bed is analyzed. According to the time of leaving the bed, it is judged whether the person has left the bed for too long. When the person has left the bed for too long, an abnormal alarm signal will be sent. When the person has left the bed for a short time, other equipment information will be searched to further obtain the specific location information of the person.

Optionally, in the method for detecting information of an object in a room provided by the embodiment of this application, the target information also includes the position information of the target object. After collecting the target information of the target object in the room by the target detection device, the method further includes: analyzing whether the target object is in bed based on the position information in the target information; when it is analyzed that the target object is in bed, judging whether the target object is in a sleep state based on the vital signs information in the target information; analyzing the heartbeat data and breathing data of the target object when it is judged that the target object is in a sleep state; generating a sleep analysis report of the target object based on the heartbeat data and respiratory data of the target object, wherein the sleep analysis includes at least the sleep quality information of the target object and the prediction information of the chronic respiratory disease of the target object.

For example, in the data storage server, the big data analysis program extracts batch information from each user's data, and the extracted information is output to another database, and the extracted data contents include the following: information of leaving bed at night, time of falling asleep, time of getting up, sleep duration, breathing, heartbeat, times of turning over, sleep apnea, etc. After all the above data features are prepared, the big data analysis program can enter feature selection and health analysis. By the above scheme, the target information including the position information of the target object can be analyzed, the sleep quality information and the prediction information of the chronic respiratory diseases of the target object can be accurately determined, and displayed on the display interface, which is convenient for users to visually check the sleep quality information and the prediction information of the chronic respiratory diseases of the target object, thereby improving the user experience and further achieving the information detection effect of indoor personnel.

As shown in FIG. 10, the location area of the target object is judged according to the data state of the radar. When the location of the target object disappears at the entrance of the bathroom, whether someone falls in the bathroom is obtained according to the fall detection radar in bathroom; when no one falls, the data of the living room detection radar is continuously obtained; when someone falls according to the living room detection radar data, the behavior data in the bathroom is analyzed, and the data of this person's fall is processed according to the analysis result. Initiate the alarm signal of the fall behavior phenomenon, save the data of this person's behavior law, analyze the behavior law in the saved data with big data, and evaluate the risk of fall and life. Among them, people or things at the target position are classified into target movement and target disappearance or quiescence. When classified into target movement, the target movement is divided into two categories according to the target type, one is to judge the current target as a person, the other is to judge the current target as an object. When the current target is a person, the vitality is estimated based on the coordinates, speed, acceleration and micro-characteristics of the person, and when the current target is an object, the object is marked according to the map database. When the target is classified as disappearing or stationary, the vital signs of this person can be obtained according to the monitoring of radar status at the disappearing point (for example, bedroom or bed), whether someone falls in the bathroom can be judged according to the monitoring of radar status at the disappearing point (for example, bathroom), and the database can be calibrated according to the monitoring of radar status at other positions at the disappearing point (for example, the main position of living room), based on the judgment of house entrance and exit, when the user goes out. Judging whether the person is resident or not based on the position of the sofa area and the activity area of the living room, when it is known that the person has no vital signs for a long time based on the judgment of the activity state, identifying the weak vital signs of the person, and initiating an alarm signal of abnormal vital signs; when it is known that the person has a falling posture based on the judgment of the activity state, the person is identified as falling, and an abnormal falling alarm signal is initiated; when it is known that the vital signs of the person are normal based on the judgment of the activity state, it is determined that the user is at rest.

Step S42, real-time data processing The back-end server receives the data output from vital signs monitoring radar once every second, including respiratory rate, heart rate, respiratory waveform, heartbeat waveform, etc. The 1 Hz timer is started in the background server, and real-time processing such as out-of-bed judgment real-time processing, apnea real-time processing, abnormal heartbeat, etc. are performed on the data according to the timer. When it is judged to be abnormal, step S43 is entered.

The data of the fall detection radar in bathroom is also called by the back-end server every 1 second to process the fall data. When it is judged that the abnormal output is valid, it will also go to step S43.

The data of radar equipment in the living room gets a packet of data every one second, and a packet of data contains four continuous time point cloud information and target trajectory information. The back-end server calls and judges the target type and target trend according to the coordinate information and the target state information, and goes to step S43 to process the result data.

Step S43, push back-end service data.

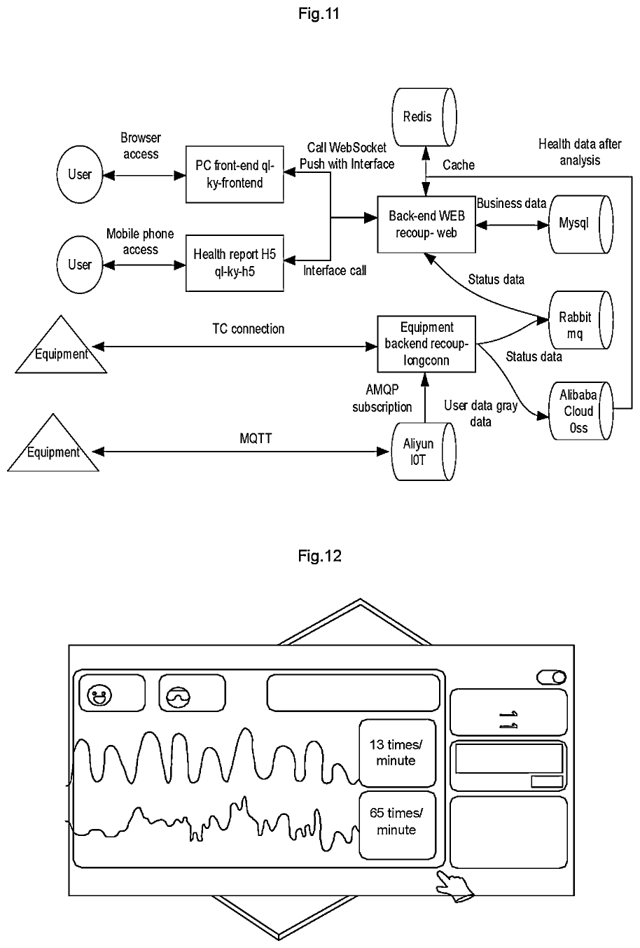

As shown in FIG. 11, the back-end server stores business data in Mysql database and caches non-relational data in Redis storage unit. The front-end server receives the result data pushed by the back-end server according to the user response of the WEB client or APP. Results by the processing flow of front-end server, the data achieved the task of step S5.

Step S5, real-time data display.

The radar data of the living room is processed in step S4, and the real-time coordinates of the person under test in the room are displayed in the interface, so that the Client Personnel can intuitively judge the state and behavior of people in the home environment.

After analyzing the behavior state information of the target object by using the behavior information in the cloud server, the method further includes: triggering the first reminding information, when the behavior state information indicates that the target object has a falling behavior phenomenon; when the behavior state information indicates that the vital signs of the target object are abnormal, triggering second reminding information; when the behavior state information indicates that the bed leaving time of the target object exceeds a preset time, triggering a third reminding message.

For example, the presentation mode of vital signs monitoring radar data is shown in FIG. 12, which can output the respiratory rate, respiratory waveform, heart rate, heartbeat waveform and sleep state of the person in the house in real time. When it is detected that the breathing rate of the person in the room is too low (that is, the vital signs are in abnormal state), a reminder alarm message (corresponding to the above-mentioned second reminder message) will be triggered.

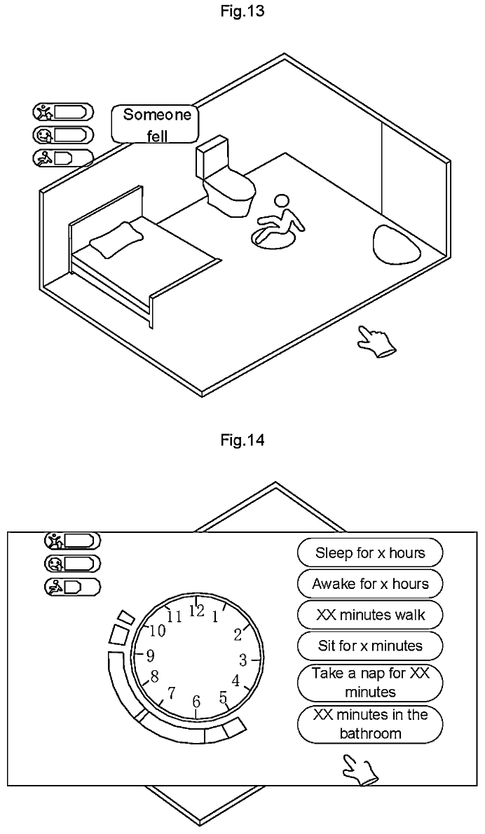

For example, as shown in FIG. 13, the display form of fall detection radar in bathroom data shows the abnormal alarm state of the actual indoor person falling in the bathroom (that is, the falling behavior phenomenon of the target object exists) by means of a cartoon character falling, and triggers an alarm reminder (corresponding to the above-mentioned first reminder information). At the same time, the client will highlight the abnormal emergency state by means of sound, so that the Client Personnel can recognize it audibly.

For example, based on the behavior state information, it is indicated that the bed leaving time of the target object exceeds the preset time (for example, the preset time is 1 hour), then an alarm reminder is triggered (corresponding to the above-mentioned third reminder information). At the same time, the client will highlight the abnormal emergency state by means of sound, so that the Client Personnel can recognize it audibly.

It should be noted that the way of the above-mentioned reminding information can be the way of triggering the alarm sound reminding or the way of triggering the staff calling the client, which is not limited in this application.

According to the scheme, the behaviors and vital signs with abnormal phenomena are triggered and reminded, so that the information of the target object can be accurately determined, and the user can be reminded in time, so that the real-time monitoring of the target object is convenient, and the user experience is improved, and the information detection effect of indoor personnel is further improved.

After collecting the target information of the target object in the room by the target detection device, the method further includes: analyzing whether the target object is in a moving state based on the behavior information in the target information; when the target object is in a moving state, determine the movement vitality information of the target object. That is, the processing after data integration pays more attention to the analysis and evaluation of the health status and vitality of people in the house, and the specific presentation mode is shown in FIG. 14. The contents of the exhibition include: statistics of sitting time, standing time, sleeping time, walking time, toilet time and frequency, etc.

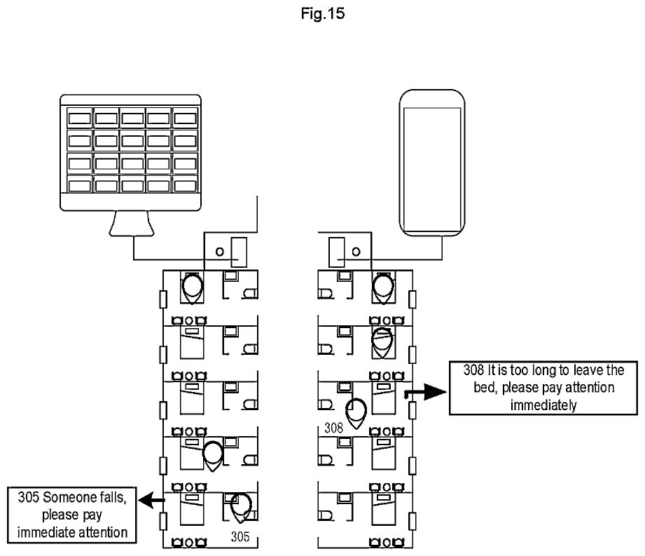

The embodiment of the application provides a data disk for all displayed information, and by the data disk, all family states where the integrated home health monitoring system is installed can be visually observed. As shown in FIG. 15, the data can be displayed on the WEB client and the mobile APP client.

Step S6, health big data analysis.

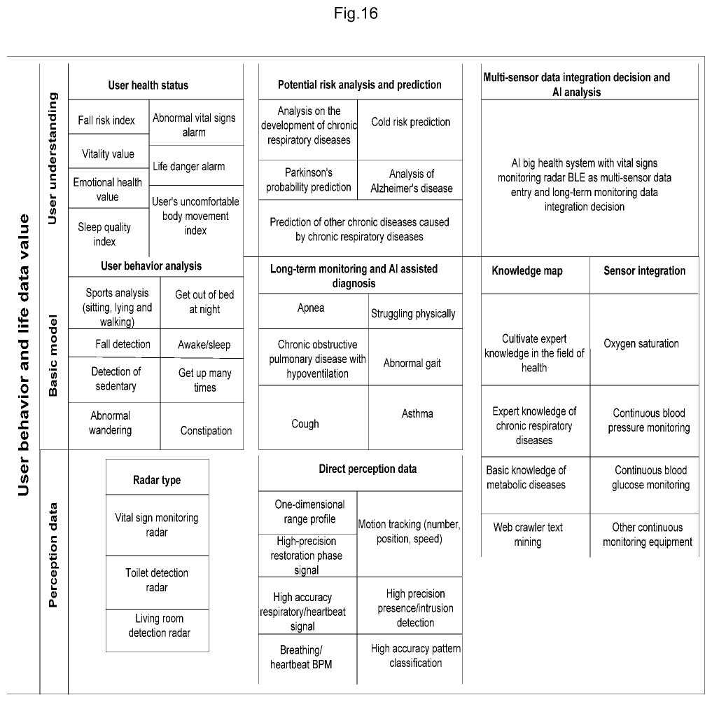

The analysis data in this step all come from the perception information output in step S41, and the specific data content is shown in FIG. 16. The data in the bottom layer in FIG. 16 is the direct perception data provided by different types of radar equipment, including one-dimensional range direction, phase signal, respiratory fluctuation signal, heartbeat fluctuation signal, respiratory rate, heart rate, personnel's motion tracking information, target intrusion and presence detection information, personnel posture and other pattern classification information. The data in the lower right corner of FIG. 16 is the integration data of blood oxygen saturation information, continuous blood pressure information, continuous blood sugar information and so on provided by other auxiliary vital signs monitoring devices.

According to the invention, user behavior analysis and AI-assisted diagnosis are carried out on multi-dimensional perception data by the basic model, and this kind of data, as the first processing data, needs to go to step S62 for data mining; combining the expert knowledge in the knowledge map with the tag data collected by the system (for example, the content of step S61), the invention can output user health status data and potential risk prediction data from a more convenient angle for users to understand.

Step S61, collecting user tags.

In this application, in the process of registering or obtaining a report on a device (such as the user's wearable device or radar monitoring device), the following information of the user: age, gender, nursing level, mobility, cardiovascular diseases, respiratory diseases, etc. is obtained by the pop-up of the client app, and this information will be stored in the data storage server as the label of the registered user.

Step S62, multi-dimensional user data mining (feature construction).

In the data storage server, the big data analysis program extracts batch information from each user's data, and the extracted information is output to another database. The extracted data content can include the following contents:
1. Information of leaving bed at night:
1) The average time for getting out of bed in the last 30 days/3 days/1 day (the embodiment of mobility, processing from one-dimensional range profile);
2) Whether the bed-leaving time has increased significantly in the last 3 days/1 day;
3) Whether the single bed-leaving time in the last 3 days/1 day exceeds 15 minutes;
2. Sleep time:
1) Sleep time for nearly 3 days/1 day;
2) The delay of falling asleep in the last 3 days/1 day compared with the average falling asleep time of the user;
3. Wake up time:
1) The time from waking up to leaving the bed of the user (this information is a part of the factors that the user is in a hurry and easily leads to accidents)
4. Sleep duration:
1) Sleep duration in the last 3 days/1 day;
2) Whether the sleep in the last 3 days/1 day is too short compared with the average of the user in the last month;
5. Breathing and heartbeat:
1) Whether breathing is abnormal in the last 3 days/1 day;
2) Whether the heart rate is abnormal in the last 3 days/1 day;
6. Turnover times:
1) Turnover frequency in the last 3 days/1 day;
2) Whether the turnover has increased significantly in the last 3 days/1 day;
7. Sleep apnea:
1) Average AHI index in the last 3 days/1 day;
2) The longest duration of apnea in the last 3 days/1 day;

After all the above data features are prepared, the big data analysis program can proceed to step S63.

Step S63, feature selection

According to the feature map and prior knowledge, the big data analysis program carries out feature correlation analysis, and removes the indistinguishable features and repetitive features with strong correlation. The input to big data analysis program is user sleep feature table and user label table. The information output by the big data analyzer is the filtered feature name.

After determining the specific feature name, the big data analysis program goes to step S64.

Step S64, health analysis model design.

Acquiring a set of target information of a target object in a preset time period from a cloud server; Screening data information of multiple dimensions from the set of target information; Based on the preset weights of each dimension and the data information of each dimension, the first health data of the target object is evaluated, wherein the health data at least comprises the body state information of the target object and the prediction information of the potential risks of the body.

The above scheme means that the big data analysis program will do the next analysis based on the filtered feature names. For example, the process can be as follows:
1) Dividing the filtered features into 5 to 7 dimensions, and setting the weight of each dimension;
2) Histogram statistics (or sorting) are carried out on the features in each dimension, and the results of sorting are mapped into scores in that dimension;
3) Sum the scores of all dimensions to get the final score of the evaluation of the user's health status and risk prediction items.

After the health big data analysis in the above steps, the health data that is easy for users and nursing staff to understand can be displayed on the client of the invention in the form of reports or pictures, for example, the first health data is displayed on the display interface according to the preset display form. So as to provide direct, reliable and valuable data for smart pension, smart city and other sectors, and empower the society.

To sum up, in the method for detecting information of an object in a room provided by the embodiment of this application, target information of target object in the room is collected by target detection equipment, wherein the target information at least comprises vital sign information and behavior information of the target object, and the target detection equipment includes radar monitoring equipment that the target object do not need to wear and vital sign monitoring equipment that the target object wears, wherein data information is transmitted between the radar monitoring equipment and the vital sign monitoring equipment by Bluetooth; based on the target information, analyze the behavior state information of the target object; displaying the behavior state information of the target object in the display interface solves the problem of poor information detection effect of indoor personnel in related technologies. By analyzing the target information including vital signs information and behavior information of the target object, the behavior state information of the target object can be accurately determined and displayed on the display interface, which is convenient for users to visually view the behavior state information of the target object, thus improving the user experience and further improving the information detection effect of indoor personnel.

It should be noted that the steps shown in the flowchart of the drawings can be executed in a computer system such as a set of computer-executable instructions, and although the logical sequence is shown in the flowchart, in some cases, the steps shown or described can be executed in a different order than here.

The embodiment of this application also provides the device for detecting information of an object in a room. It should be noted that the device for detecting information of an object in a room of this application embodiment can be used to execute the method for detecting information of an object in a room provided by this application embodiment. The device for detecting information of an object in a room provided by the embodiment of this application will be described below.

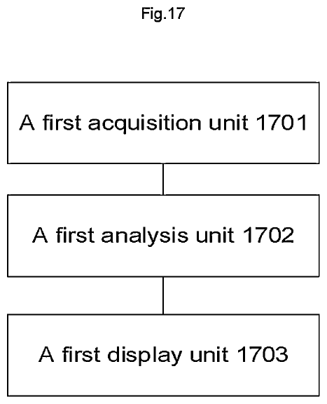

FIG. 17 is a schematic diagram of the device for detecting information of an object in a room according to an embodiment of the present application. As shown in FIG. 17, the device includes a first collection unit 1701, a first analysis unit 1702 and a first display unit 1703.

Specifically, the first collecting unit 1701 is configured to collect target information of target object in the room by target detection equipment, wherein the target information at least comprises vital sign information and behavior information of the target object, and the target detection equipment includes radar monitoring equipment that the target object do not need to wear and vital sign monitoring equipment that the target object wears, wherein data information is transmitted between the radar monitoring equipment and the vital sign monitoring equipment by Bluetooth;

The first analysis unit 1702 is configured to analyze the behavior state information of the target object based on the target information;

The first display unit 1703 is configured to display the behavior state information of the target object on the display interface.

To sum up, in the device for detecting information of an object in a room provided by the embodiment of this application, the first acquisition unit 1701 collects target information of target object in the room by target detection equipment, wherein the target information at least comprises vital signs information and behavior information of the target object, and the target detection equipment includes radar monitoring equipment that the target object do not need to wear and vital signs monitoring equipment that the target object wears, wherein data information is transmitted between the radar monitoring equipment and the vital signs monitoring equipment by Bluetooth; the first analysis unit 1702 analyzes the behavior state information of the target object based on the target information; the first display unit 1703 displays the behavior state information of the target object on the display interface, which solves the problem of poor information detection effect of indoor personnel in related technologies. By analyzing the target information including vital signs information and behavior information of the target object, the behavior state information of the target object can be accurately determined and displayed on the display interface, which is convenient for users to visually view the behavior state information of the target object, thereby improving the user experience and further improving the information detection effect of indoor personnel.

Optionally, in the device for detecting information of an object in a room provided by the embodiment of this application, the device further comprises: a first arranging unit configured to set a plurality of radar monitoring devices indoors before collecting target information of target object in the room by target detection devices, wherein, the plurality of radar monitoring devices are configured to collecting falling behavior information and indoor behavior information of target objects; a first determination unit configured to take a plurality of radar devices as devices in the target detection equipment.

Optionally, in the device for detecting information of an object in a room provided by the embodiment of this application, the device further comprises: a first uploading unit configured to upload the target information of target object in the room to the cloud server by the WIFI module and/or the Bluetooth module after collecting the target information of target object in the room by the target detection device; the first analysis unit, configured to analyze the target object based on the target information in the cloud server to obtain the behavior state information of the target object.

Optionally, in the device for detecting information of an object in a room provided by the embodiment of this application, the target information also includes the location information of the target object, and the third analysis unit includes: a first analysis module configured to analyze the vital sign information and behavior state information of the target object in the cloud server by using the behavior information when the location information of the target object indicates that the target object is in the indoor bedroom bed; a second analysis module, configured to analyze the behavior state information of the target object in the cloud server by using the behavior information when the position information of the target object indicates that the target object is in the indoor bathroom; a third analysis module, configured to analyze the behavior state information of the target object in the cloud server by using vital signs information when the location information of the target object indicates that the target object is in the living room indoors.

Optionally, in the device for detecting information of an object in a room provided by the embodiment of this application, the device further comprises: a first triggering unit configured to trigger the first reminding information when the behavior information indicates that the target object has a falling behavior phenomenon after analyzing the behavior information of the target object in the cloud server; a second triggering unit configured to trigger second reminding information when the behavior state information indicates that the vital signs of the target object are abnormal; a third triggering unit, configured to trigger a third reminding message when the behavior state information indicates that the bed leaving time of the target object exceeds a preset time.

Optionally, in the device for detecting information of an object in a room provided by the embodiment of this application, the device further comprises a first acquisition unit configured to acquire a set of target information of target objects in a preset time period from a cloud server; a first filtering unit configured to filter out data information of multiple dimensions from the set of target information; a first evaluation unit configured to evaluate the first health data of the target object based on the preset weights of each dimension and the data information of each dimension, wherein the health data at least comprises the body state information of the target object and the prediction information of the potential risks of the body.

Optionally, in the device for detecting information of an object in a room provided by the embodiment of this application, the device further comprises a second display unit, which is configured to display the first health data on the display interface according to a preset display form after evaluating the health status and body risk of the target object based on the preset weights of each dimension and the data information of each dimension.

Optionally, in the device for detecting information of an object in a room provided by the embodiment of this application, the device further comprises a fourth analysis unit, which is arranged to analyze whether the target object is in a moving state based on the behavior information in the target information after collecting the target information of the target object in the room by the target detection device; a first determining unit configured to determine the movement vitality information of the target object when the target object is in a moving state.

Optionally, in the device for detecting information of an object in a room provided by the embodiment of this application, the target information further includes the position information of the target object, and the device further includes a fifth analysis unit configured to analyze whether the target object is in bed based on the position information in the target information after collecting the target information of the target object in the room by the target detection device; a first judging unit configured to judge whether the target object is in a sleep state based on vital signs information in the target information when it is analyzed that the target object is in bed; a sixth analysis unit configured to analyze the heartbeat data and respiratory data of the target object when it is judged that the target object is in a sleep state; a first generating unit configured to generate a sleep analysis report of the target object based on the heartbeat data and respiratory data of the target object, wherein the sleep analysis includes at least the sleep quality information of the target object and the prediction information of the chronic respiratory disease of the target object.

The device for detecting information of an object in a room includes a processor and a memory. The first acquisition unit 1701, the first analysis unit 1702 and the first display unit 1703 are stored in the memory as program units, and the processor executes the program units stored in the memory to realize corresponding functions.

The processor contains a kernel, and the kernel goes to the memory to retrieve the corresponding program unit. One or more kernels can be set, and the information detection effect of indoor personnel can be improved by adjusting kernel parameters.

The memory may include nonvolatile memory, random access memory (RAM) and/or nonvolatile memory in computer readable media, such as read-only memory (ROM) or flash memory (flash RAM), and the memory includes at least one memory chip.

The embodiment of the invention provides a storage medium on which a program is stored, which, when executed by a processor, realizes the information detection method of the indoor object.

The embodiment of the invention provides a processor for running a program, wherein the information detection method of the indoor object is executed when the program runs.

The embodiment of the invention provides a device, which comprises a processor, a memory and a program stored in the memory and running on the processor. When the processor executes the program, it realizes the following steps: collecting target information of target object in the room by a target detection device, wherein the target information at least comprises vital sign information and behavior information of the target object, the target detection equipment includes radar monitoring equipment that the target object does not need to wear and vital signs monitoring equipment that the target object wears, wherein data information is transmitted between the radar monitoring equipment and the vital signs monitoring equipment by Bluetooth; analyzing the behavior state information of the target object based on the target information; displaying the behavior state information of the target object on the display interface.

When the processor executes the program, it also realizes the following steps: before collecting the target information of the target object in the room by the target detection device, the method also includes: arranging a plurality of radar monitoring devices in the room, wherein the plurality of radar monitoring devices are configured to collecting the falling behavior information and indoor behavior information of the target object; The plurality of radar devices as devices in the target detection equipment.

When the processor executes the program, it also realizes the following steps: after collecting the target information of the target object in the room by the target detection device, the method also includes uploading the target information to the cloud server by the WIFI module; analyzing the behavior state information of the target object based on the target information includes analyzing the target object based on the target information in the cloud server to obtain the behavior state information of the target object.

When the processor executes the program, it also realizes the following steps: the target information also includes the location information of the target object; in the cloud server, the target object is analyzed based on the target information to obtain the behavior state information of the target object; when the location information of the target object indicates that the target object is in an indoor bedroom bed, the behavior information is used in the cloud server to analyze the vital sign information and behavior state information of the target object; when the position information of the target object indicates that the target object is in the indoor bathroom, analyzing the behavior state information of the target object by using the behavior information in the cloud server; when the location information of the target object indicates that the target object is in the indoor living room, the vital sign information is used in the cloud server to analyze the behavior state information of the target object.

When the processor executes the program, it also realizes the following steps: after analyzing the behavior state information of the target object by using the behavior information in the cloud server, the method also includes: when the behavior state information indicates that the target object has a falling behavior phenomenon, triggering the first reminding information; when the behavior state information indicates that the vital signs of the target object are abnormal, triggering second reminding information.

When the processor executes the program, it also realizes the following steps: the method also includes: acquiring a set of target information of the target object in a preset time period from a cloud server; screening data information of multiple dimensions from the set of target information; evaluating the first health data of the target object based on the preset weight of each dimension and the data information of each dimension, wherein the health data at least comprises the body state information of the target object and the prediction information of the body potential risk.

When the processor executes the program, it also realizes the following steps: after evaluating the health status and body risk of the target object based on the preset weight of each dimension and the data information of each dimension, the method further includes: displaying the first health data on the display interface according to the preset display form.

When the processor executes the program, it also realizes the following steps: after collecting the target information of the target object in the room by the target detection device, the method also includes: analyzing whether the target object is in a moving state based on the behavior information in the target information; when the target object is in a moving state, the movement vitality information of the target object is determined.

When the processor executes the program, it also realizes the following steps: the target information also includes the position information of the target object; after collecting the target information of the target object in the room by the target detection device, the method also includes: analyzing whether the target object is in bed or not based on the position information of the target information; when it is analyzed that the target object is in bed, judging whether the target object is in a sleep state based on vital sign information in the target information; analyzing the heartbeat data and breathing data of the target object when it is judged that the target object is in a sleep state; generating a sleep analysis report of the target object based on the heartbeat data and respiratory data of the target object, wherein the sleep analysis includes at least the sleep quality information of the target object and the prediction information of the chronic respiratory disease of the target object. The devices in this paper can be servers, PC, PAD, cell phones, etc.

The application also provides a computer program product, which, when executed on data processing equipment, is suitable for executing a program initialized with the following method steps: collecting target information of target object in the room by target detection equipment, wherein the target information at least comprises vital sign information and behavior information of the target object, and the target detection equipment includes radar monitoring equipment that the target object do not need to wear and vital sign monitoring equipment that the target object wears; analyzing the behavior state information of the target object based on the target information; Displaying the behavior state information of the target object on the display interface.

When executed on the data processing equipment, it is also suitable for executing the program initialized with the following method steps: before collecting the target information of target object in the room by the target detection equipment, the method further comprises: arranging a plurality of radar monitoring devices in the room, wherein the plurality of radar monitoring devices are configured to collecting the falling behavior information and indoor behavior information of the target object; the plurality of radar devices as devices in the target detection equipment.

When executed on the data processing device, it is also suitable for executing the program initialized with the following method steps: after collecting the target information of target object in the room by the target detection device, the method further includes uploading the target information to the cloud server by the WIFI module; analyzing the behavior state information of the target object based on the target information includes analyzing the target object based on the target information in the cloud server to obtain the behavior state information of the target object.

When executed on a data processing device, it is also suitable for executing a program initialized with the following method steps: the target information also includes the location information of the target object; in the cloud server, analyzing the target object based on the target information to obtain the behavior state information of the target object includes: when the location information of the target object indicates that the target object is in the indoor bathroom, using the behavior information to analyze the behavior state information of the target object in the cloud server; when the location information of the target object indicates that the target object is in the indoor living room, the vital sign information is used in the cloud server to analyze the behavior state information of the target object.

When executed on the data processing equipment, it is also suitable for executing the program initialized with the following method steps: after analyzing the behavior state information of the target object by using the behavior information in the cloud server, the method further comprises: triggering the first reminding information when the behavior state information indicates that the target object has a falling behavior phenomenon; when the behavior state information indicates that the vital signs of the target object are abnormal, triggering second reminding information; when the behavior state information indicates that the bed leaving time of the target object exceeds a preset time, triggering a third reminding message.

When executed on data processing equipment, it is also suitable for executing the program initialized with the following method steps: the method also includes: acquiring the set of target information of the target object in a preset time period from a cloud server; screening data information of multiple dimensions from the set of target information; evaluating the first health data of the target object based on the preset weight of each dimension and the data information of each dimension, wherein the health data at least comprises the body state information of the target object and the prediction information of the body potential risk.

When executed on the data processing device, it is also suitable for executing the program initialized with the following method steps: after evaluating the health status and body risk of the target object based on the preset weights of each dimension and data information of each dimension, the method further comprises: displaying the first health data on the display interface according to a preset display form.

When executed on the data processing device, it is also suitable for executing the program initialized with the following method steps: after collecting the target information of the target object in the room by the target detection device, the method further comprises: analyzing whether the target object is in a moving state based on the behavior information in the target information; when the target object is in a moving state, the movement vitality information of the target object is determined.

When executed on the data processing device, it is also suitable for executing the program initialized with the following method steps: the target information also includes the position information of the target object; after collecting the target information of the target object in the room by the target detection device, the method further includes: analyzing whether the target object is in bed based on the position information in the target information; when it is analyzed that the target object is in bed, judging whether the target object is in a sleep state based on vital sign information in the target information; analyzing the heartbeat data and breathing data of the target object when it is judged that the target object is in a sleep state; generating a sleep analysis report of the target object based on the heartbeat data and respiratory data of the target object, wherein the sleep analysis includes at least the sleep quality information of the target object and the prediction information of the chronic respiratory disease of the target object.

It should be understood by those skilled in the art that the embodiments of this application can be provided as a method, a system, or a computer program product. Therefore, this application can take the form of an entirely hardware embodiment, an entirely software embodiment or an embodiment combining software and hardware aspects. Furthermore, the application can take the form of a computer program product implemented on one or more computer usable storage media (including but not limited to disk memory, CD-ROM, optical memory, etc.) having computer usable program codes embodied therein.

The application is described with reference to flow charts and/or block diagrams of methods, devices (systems), and computer program products according to embodiments of the application. It should be understood that each flow and/or block in the flow charts and/or block diagrams, as well as combinations of flows and/or blocks in the flow charts and/or block diagrams can be implemented by computer program instructions. These computer program instructions can be provided to the processor of a general-purpose computer, a special-purpose computer, an embedded processor or other programmable data processing equipment to produce a machine, so that the instructions executed by the processor of the computer or other programmable data processing equipment produce means for realizing the functions specified in one or more flow charts and/or one or more block diagrams.

These computer program instructions can also be stored in a computer-readable memory that can guide a computer or other programmable data processing equipment to work in a specific way, so that the instructions stored in the computer-readable memory produce an article of manufacture including instruction devices that implement the functions specified in one or more flow charts and/or one or more block diagrams.

These computer program instructions can also be loaded on a computer or other programmable data processing equipment, so that a series of operation steps are executed on the computer or other programmable equipment to generate computer-implemented processing, so that the instructions executed on the computer or other programmable equipment provide steps for realizing the functions specified in one or more flows of the flowchart and/or one or more blocks of the block diagram.

In a typical configuration, a computing device includes one or more processors (CPU), input/output interfaces, network interfaces and memory.

Memory may include non-permanent memory, random access memory (RAM) and/or non-volatile memory in computer-readable media, such as read-only memory (ROM) or flash RAM. Memory is an example of a computer readable medium.

Computer-readable media include permanent and non-permanent, removable and non-removable media. Information storage can be realized by any method or technology. The information can be computer-readable instructions, data structures, modules of programs or other data. Examples of storage media of computers include, but not limited to phase change memory (PRAM), static random access memory (SRAM), dynamic random access memory (DRAM), other types of random access memory (RAM), read-only memory (ROM), electrically erasable programmable read-only memory (EPROM), flash memory or other memory technologies, CD-ROM, digital versatile disk (DVD) Or other optical storage, magnetic cassette tape, magnetic magnetic disk storage or other magnetic storage devices or any other non-transmission medium, which can be used to store information that can be accessed by computing devices. As defined in this paper, computer-readable media does not include temporary computer-readable media, such as modulated data signals and carrier waves.

It should also be noted that the terms “including”, “including” or any other variant thereof are intended to cover non-exclusive inclusion, so that a process, method, commodity or equipment that includes a series of elements includes not only those elements, but also other elements not explicitly listed, or elements inherent to such process, method, commodity or equipment. Without further restrictions, the element defined by the sentence “including a . . . ” does not exclude that there are other identical elements in the process, method, commodity or equipment including the element.

It should be understood by those skilled in the art that the embodiments of this application can be provided as a method, a system or a computer program product. Therefore, this application can take the form of an entirely hardware embodiment, an entirely software embodiment or an embodiment combining software and hardware aspects. Furthermore, the application can take the form of a computer program product implemented on one or more computer usable storage media (including but not limited to disk memory, CD-ROM, optical memory, etc.) having computer usable program codes embodied therein.

The above are only examples of this application, and are not intended to limit this application. Various modifications and changes can be made in this application to those skilled in the art. Any modification, equivalent substitution, improvement, etc. made within the spirit and principle of this application should be included within the scope of the claims of this application.

1. A method for detecting information of an object in a room, comprising:
collecting target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth;
analyzing behavior state information of the target object based on the target information; and
displaying the behavior state information of the target object on a display interface.

2. The method according to claim 1, wherein before collecting the target information of the target object in the room by the target detection equipment, the method further comprising:
arranging a plurality of radar monitoring devices in the room, wherein the plurality of radar monitoring devices are configured to collect falling behavior information and indoor behavior information of the target object; and
taking the plurality of radar monitoring devices as devices in the target detection equipment.

3. The method according to claim 1, wherein after collecting target information of a target object in the room by target detection equipment, the method further comprises:
uploading the target information to a cloud server by a WIFI module;
based on the target information, analyzing the behavior state information of the target object includes:
in the cloud server, analyzing the target object based on the target information to obtain the behavior state information of the target object.

4. The method according to claim 3, wherein the target information also includes the location information of the target object, and in the cloud server, analyzing the target object based on the target information to obtain the behavior state information of the target object comprises:
when the location information of the target object indicates that the target object is in the indoor bedroom bed, analyzing the vital sign information and behavior state information of the target object by using the behavior information in the cloud server;
when the position information of the target object indicates that the target object is in the indoor bathroom, analyzing the behavior state information of the target object by using the behavior information in the cloud server; and
when the location information of the target object indicates that the target object is in the indoor living room, the vital sign information is used in the cloud server to analyze the behavior state information of the target object.

5. The method according to claim 4, wherein after analyzing the behavior state information of the target object by using the behavior information in the cloud server, the method further comprises:
triggering first reminding information when the behavior state information indicates that the target object has a falling behavior phenomenon;
when the behavior state information indicates that the vital signs of the target object are abnormal, triggering second reminding information; and
when the behavior state information indicates that the bed leaving time of the target object exceeds a preset time, triggering a third reminding message.

6. The method according to claim 3, further comprising:
acquiring a set of target information of the target object in a preset time period from a cloud server;
screening data information of multiple dimensions from the set of target information; and
evaluating the first health data of the target object based on the preset weight of each dimension and the data information of each dimension, wherein the health data at least comprises the body state information of the target object and the prediction information of the body potential risk.

7. The method according to claim 6, wherein after evaluating the health status and body risk of the target object based on the preset weight of each dimension and the data information of each dimension, the method further comprises:
displaying the first health data on the display interface according to a preset display form.

8. The method according to claim 1, wherein after target information of a target object in the room is collected by target detection equipment, the method further comprises:
analyzing whether the target object is in a moving state based on the behavior information in the target information; and
when the target object is in a moving state, the movement vitality information of the target object is determined.

9. The method according to claim 1, wherein the target information also includes the position information of the target object, and after the target information of the target object in the room is collected by the target detection equipment, the method further comprises:
analyzing whether the target object is on the bed based on the position information in the target information;
when it is analyzed that the target object is in bed, judging whether the target object is in a sleep state based on vital sign information in the target information;
analyzing the heartbeat data and breathing data of the target object when it is judged that the target object is in a sleep state; and
generating a sleep analysis report of the target object based on the heartbeat data and respiratory data of the target object, wherein the sleep analysis includes at least the sleep quality information of the target object and the prediction information of the chronic respiratory disease of the target object.

10. A device for detecting information of an object in a room, comprising:
a first collecting unit, configured to collect target information of a target object in the room by target detection equipment, wherein the target information at least comprises vital sign information and behavior information of the target object, and the target detection equipment comprises radar monitoring equipment that the target object do not need to wear and vital sign monitoring equipment that the target object wears, wherein data information is transmitted between the radar monitoring equipment and the vital sign monitoring equipment by Bluetooth;
a first analysis unit, configured to analyze the behavior state information of the target object based on the target information; and
a first display unit, configured to display the behavior state information of the target object on a display interface.

11. The device according to claim 10, further comprising:
a first arranging unit, configured to arrange a plurality of radar monitoring devices in the room, before collecting the target information of the target object in the room by the target detection equipment, wherein the plurality of radar monitoring devices are configured to collect the falling behavior information and indoor behavior information of the target object;
a first determination unit, configured to take the plurality of radar monitoring devices as devices in the target detection equipment.

12. The device according to claim 10, wherein the device further comprises:
the first uploading unit, configured to upload the target information of target object in the room to the cloud server by the WIFI module after collecting the target information of target object in the room by the target detection device; and
a first analysis unit, configured to analyze the target object based on the target information in the cloud server to obtain the behavior state information of the target object.

13. The device according to claim 12, wherein the target information further includes position information of the target object, and the third analysis unit comprises:
a first analysis module, configured to analyze the vital sign information and behavior state information of the target object in the cloud server by using the behavior information when the location information of the target object indicates that the target object is in the indoor bedroom bed;
a second analysis module, configured to analyze the behavior state information of the target object in the cloud server by using the behavior information when the location information of the target object indicates that the target object is in the indoor bathroom; and
a third analysis module, configured to analyze the behavior state information of the target object in the cloud server by using the vital signs information when the location information of the target object indicates that the target object is in the living room indoors.

14. The device according to claim 13, further comprising:
a first triggering unit, configured to trigger first reminding information when the behavior state information indicates that the target object has a falling behavior phenomenon after analyzing the behavior state information of the target object by using the behavior information in the cloud server;
a second triggering unit, configured to trigger second reminding information when the behavior state information indicates that the vital signs of the target object are abnormal; and
a third triggering unit, configured to trigger a third reminding message when the behavior state information indicates that the bed leaving time of the target object exceeds a preset time.

15. The device according to claim 12, further comprising:
a first acquisition unit, configured to acquire a set of target information of the target object in a preset time period from a cloud server;
a first filtering unit, configured to filter out data information of multiple dimensions from the set of target information; and
a first evaluation unit, configured to evaluate the first health data of the target object based on the preset weights of each dimension and the data information of each dimension, wherein the health data at least comprises the body state information of the target object and the prediction information of the potential risks of the body.

16. The device according to claim 15, further comprising:
a second display unit, configured to display the first health data on the display interface according to a preset display form after evaluating the health status and body risk of the target object based on the preset weight of each dimension and data information of each dimension.

17. The device according to claim 10, further comprising:
the fourth analysis unit, configured to analyze whether the target object in the room is in a moving state based on the behavior information in the target information after the target information of the target object in the room is collected by the target detection device; and
a first determining unit, configured to determine the movement vitality information of the target object when the target object is in a moving state.

18. The device according to claim 10, wherein the target information further includes the location information of the target object, and the device further comprises:
a fifth analysis unit, configured to analyze whether the target object is on the bed based on the position information in the target information after collecting the target information of the target object in the room by the target detection device;
a first judging unit, configured to judge whether the target object is in a sleep state based on vital signs information in the target information when it is analyzed that the target object is in bed;
a sixth analysis unit, configured to analyze the heartbeat data and respiratory data of the target object when it is judged that the target object is in a sleep state; and
a first generating unit, configured to generate a sleep analysis report of the target object based on the heartbeat data and respiratory data of the target object, wherein the sleep analysis includes at least the sleep quality information of the target object and the prediction information of the chronic respiratory disease of the target object.

19. A processor for running a program, wherein the program executes the method for detecting information of an object in a room according to claim 1.

20. A storage medium comprising a stored program, wherein the program executes the method for detecting information of an object in a room according to claim 1.
